(19) United States
(12) Patent Application Publication
(10) Pub. No.: US 2014/0276991 A1
(43) Pub. Date: Sep. 18, 2014

(54) GASTRIC PLICATION GUIDE

(71) Applicant: THE CLEVELAND CLINIC FOUNDATION, Cleveland, OH (US)

(72) Inventor: Philip Schauer, Hunting Valley, OH (US)

(73) Assignee: THE CLEVELAND CLINIC FOUNDATION, Cleveland, OH (US)

(21) Appl. No.: 14/202,598

(22) Filed: Mar. 10, 2014

Related U.S. Application Data
(60) Provisional application No. 61/791,880, filed on Mar. 15, 2013.

(51) Int. Cl. A61F 5/00; A61B 17/04

(52) U.S. Cl. CPC A61F 5/0086; A61B 17/0482
USPC 606/148

(57) ABSTRACT

A device for helping a surgeon to fold the correct amount of tissue when conducting a gastric plication procedure is described. The guide device includes an elongated flexible guide, a securing device positioned on the distal end of the guide, and means for guiding stomach tissue during a gastric plication procedure that are positioned longitudinally along the distal end of the guide and proximal to the securing device. The means for guiding stomach tissue include the use of a shaping balloon to serve as a guide for final stomach volume and/or a groove on the guide device to regulate formation of the tissue fold. Methods for using the guide device to carry out gastric plication procedures are also described.

### RELATED APPLICATIONS

This application claims the benefit of U.S. Provisional Patent Application Ser. No. 61/791,880, filed Mar. 15, 2013, the entirety of which is hereby incorporated by reference in its entirety for all purposes.

### BACKGROUND

There are currently about 225,000 bariatric surgeries carried out in the U.S. each year, with a roughly equal number being carried out outside of the U.S. Bariatric surgery is prescribed to treat morbid obesity (defined as a body mass index greater than 40), type 2 diabetes, hypertension, sleep apnea, and other co-morbid conditions. The majority of these surgeries are gastric bypass, with the remaining being a fairly even mix of sleeve gastrectomy and gastric banding procedures.

Gastric bypass (GB) surgery divides the stomach into a small upper pouch and a much larger lower “remnant” pouch and then re-arranges the small intestine to connect to both. GB surgery leads to a marked reduction in the functional volume of the stomach, accompanied by an altered physiological and physical response to food. However, a recent study revealed that 15% of patients experience complications as a result of gastric bypass, and 0.5% of patients died within six months of GB surgery due to complications.

Gastric banding and sleeve gastrectomy are two additional bariatric surgical procedures that have been developed. In gastric banding, an inflatable band is placed around the upper part of the stomach to create a smaller stomach pouch. Gastric banding is the least invasive type of bariatric surgery, but its efficacy has recently been called into question. Sleeve gastrectomy is a surgical weight-loss procedure particularly useful for extremely obese patients in which the stomach is reduced to about 25% of its original size, by surgical removal of a large portion of the stomach, following the major curve. The open edges of the stomach are then attached together to form a sleeve or tube. However, because sleeve gastrectomy permanently removes a portion of the stomach, it is not reversible.

Gastric plication is a variation of the sleeve gastrectomy procedure in which a portion of the stomach is folded in upon itself. Because a portion of the stomach is folded rather than removed, the procedure is reversible. However, if too much tissue is folded in, food cannot pass through the stomach. On the other hand, if insufficient tissue is folded in, the patient will not lose enough weight. Accordingly, it is very important to be precisely control stomach size when conducting a gastric plication procedure.

### SUMMARY

A gastric plication guide is described herein that helps a surgeon fold in the correct amount of tissue when carrying out a gastric placation procedure to help the patient lose weight without blocking the stomach.

In one aspect, a gastric plication guide device is provided that includes an elongated flexible guide, a securing device positioned on the distal end of the guide, and means for guiding stomach tissue during a gastric plication procedure, positioned longitudinally along the distal end of the guide and proximal to the securing device. Various means for guiding stomach tissue are provided. In one embodiment, the means for guiding stomach tissue comprise a shaping balloon. In some embodiments, the shaping balloon is used alone, while in other embodiments the shaping balloon includes additional smaller balloons to further assist in formation of the tissue fold. In further embodiments, the flexible guide includes a lumen including one or more ports in communication with the lumen to apply suction during the procedure. In yet further embodiments, the means for guiding stomach tissue include a groove sized to accommodate folded stomach tissue.

Another aspect of the invention provides methods for using the gastric plication guide devices described herein to carry out a gastric plication procedure.

### BRIEF DESCRIPTION OF THE FIGURES

FIG. 1 provides a cross-sectional view of the prior art method of gastric plication, with 1(a) showing a side view of the stomach, FIG. 1(b) showing an x-plane cross-sectional view of the stomach before the procedure has begun with the suture points marked, FIG. 1(c) showing an x-plane cross-sectional view of the stomach after the initial tissue fold has been formed by the first line of sutures, and FIG. 1(d) showing an x-plane cross-sectional view of the stomach after the full tissue fold has been formed and the second line of sutures have been placed.

FIG. 2 provides (a) a view of the gastric plication guide including a shaping balloon and suction ports while (b) provides a y-plane cross-sectional view of the gastric plication guide including a shaping balloon.

FIG. 3 provides a side view of a stomach in which the gastric plication guide has been positioned at the outset of a gastric plication procedure.

FIG. 4 provides x-plane cross sectional views of a stomach showing three steps of a gastric plication procedure using a gastric plication guide including a shaping balloon and suction ports, with the first step (a) showing the gastric plication guide that has been adhered to the side of the stomach by suction through the ports at the beginning of the procedure, a second step (b) showing the partial inflation of the balloon, formation of the initial tissue fold, and application of the first line of sutures, and a third step (c) showing full inflation of the balloon, formation of the full tissue fold, and application of the second line of sutures.

FIG. 5 provides a schematic view of the steps involved in using a gastric plication guide including suction ports and a shaping balloon to carry out a gastric plication procedure.

FIG. 6 provides cross-sectional views of a gastric plication procedure in which three additional balloons are together with the shaping balloon to assist in formation of the tissue fold, with the first step (a) showing inflation of the central and peripheral guide balloons to induce formation of a tissue fold, second step (b) showing further expansion of the peripheral guide balloons to capture the tissue fold and application of the first line of sutures, and (c) deflation of the central guide balloon and partial deflation of the peripheral guide balloons to allow further development of the tissue fold and application of the second line of sutures upon re-inflation of the peripheral guide balloons to re-capture the tissue fold.

FIG. 7 provides a schematic view of the steps involved in a gastric plication procedure using additional guide balloons.

FIG. 8 provides views of gastric placation guides including a tissue groove, with (a) providing a view of the gastric plication guide with a tissue groove, (b) providing the associated y-plane cross-sectional view of the gastric plication guide with a tissue groove, (c) providing a view of a gastric plication guide with a tissue groove including embedded suction ports and (d) providing an associated y-plane cross-sectional view of a gastric plication guide with a tissue groove including embedded suction ports.

FIG. 9 provides a schematic view of the steps involved in a gastric plication procedure including a groove to accommodate formation of the tissue fold.

The following detailed description is to be read with reference to the figures, in which like elements in different figures have like reference numerals. The figures, which are not necessarily to scale, depict selected embodiments and are not intended to limit the scope of the invention. Skilled artisans will recognize the embodiments provided herein have many useful alternatives that fall within the scope of the invention.

### DETAILED DESCRIPTION

Unless otherwise defined, all technical and scientific terms used herein have the same meaning as commonly understood by one of ordinary skill in the art to which this application pertains. Although methods and materials similar or equivalent to those described herein can be used in the practice or testing of the exemplary embodiments, suitable methods and materials are described below. In case of conflict, the present specification, including definitions, will control. In addition, the materials, methods, and examples are illustrative only and not intended to be limiting.

### DEFINITIONS

The terminology as set forth herein is for description of the embodiments only and should not be construed as limiting the application as a whole. Unless otherwise specified, “a,” “an,” “the,” and “at least one” are used interchangeably. Furthermore, as used in the description of the application and the appended claims, the singular forms “a”, “an”, and “the” are inclusive of their plural forms, unless contraindicated by the context surrounding such.

The recitations of numerical ranges by endpoints include all numbers subsumed within that range (e.g., 1 to 5 includes 1, 1.5, 2, 2.75, 3, 3.80, 4, 5, etc.).

It is understood that all spatial references, such as “horizontal,” “vertical,” “top,” “upper,” “lower,” “bottom,” “left,” and “right,” are for illustrative purposes only and can be varied within the scope of the disclosure.

A prior art method for carrying out a gastric plication procedure is shown in FIG. 1(a-d). FIG. 1(a) shows a side view of the stomach 10, which is a generally tubular organ. The esophagus 12 leads to the stomach 10, while the small intestine 14 leads away from the stomach 10. The pyloric valve 16 is positioned between the stomach 10 and the small intestine 14. FIGS. 1(b-d) provide an X-plane cross-section view of the stomach shown in FIG. 1a. FIG. 1(b) shows the stomach 10 and the initial suture points 18 and the final suture points 20. A gastric plication procedure is done by first adjoining stomach tissue by suturing it together at the initial suture points 18, as shown in 1(c), thereby forming a first line of sutures 22 and an initial tissue fold 24. The initial tissue fold (i.e., an intraluminal fold) is formed by taking a portion of the stomach (e.g., of the anterior gastric wall) and folding it inwards (e.g., from the fundus to the antrum), and runs along a substantial portion of the length of the stomach. An example of a suitable suture is a 2-0 polypropylene running suture. This process is then repeated as shown in FIG. 1(d) by increasing the size of the tissue fold 24 to form a final tissue fold by adjoining and suturing the stomach tissue together at the final suture points 20 using a second line of sutures 26. As can be seen on the figure, the first line of sutures 22 end up around the middle of the final tissue fold, and well within the stomach. The sutures can be placed via a laparoscopic procedure. A detailed description of the laparoscopic version of the procedure is described by Brethauer et al., and is incorporated in its entirety by reference in herein. Brethauer et al., Surg Obes Relat Dis., 7(1): 15-22 (2011).

Disclosed herein are a variety of gastric plication guides that help a surgeon fold in the correct amount of tissue and/or govern the size of a stomach altered by the gastric plication procedure to help the patient lose weight without blocking the stomach. Accordingly, in one aspect, the present invention provides a gastric plication guide device that includes an elongated flexible guide 32, a securing device 34 positioned on the distal end of the guide, and means for guiding stomach tissue during a gastric plication procedure, positioned longitudinally along the distal end of the guide 32 and proximal to the securing device 34. The guide need be flexible only to the extent to allow it to be maneuvered into the stomach and to allow proper positioning therein. The securing device can be a balloon or a thick sleeve that helps to position the gastric plication guide 30 within the stomach 10, generally by resting against the pyloric valve 16. Guiding, as used herein, refers to any positioning assistance for formation of the tissue fold within the stomach or for setting the final size of the stomach after completion of the procedure.

In some embodiments, the gastric plication guide includes a shaping balloon 36. The shaping balloon 36 should be made from a suitable material such as rubber or latex, and will have a shape suitable to conform to the interior of the stomach (i.e., generally ovoid or cylindrical). The shaping balloon 36 should also be connected to an air source through an air line so that the balloon can be inflated and deflated during the gastric plication procedure. The shaping balloon 36 can be used to regulate the final size of the stomach, and/or to assist in creating a tissue fold 24 having a suitable size. As used herein, full inflation refers to inflating the balloon to its largest intended size in the context of the surgical procedure, partial inflation refers to less than full inflation, but enough inflation to substantially increase the size of the balloon (e.g., to 25%, 50%, or 75% of its final fully inflated size), and deflation refers to returning the balloon to its original size, except for partial deflation, which provides a balloon having the same sizes described for a partially inflated balloon.

An embodiment including a shaping balloon 36 is shown in FIG. 2. In the particular embodiment shown, the shaping balloon 36 is positioned along one side of the gastric plication guide 30. In this embodiment, the flexible guide 32 of the gastric plication guide 30 includes a lumen 40 and one or more ports 38 in communication with the lumen 40 positioned along the guide 32 opposite from the shaping balloon 36. The ports 38 can be used to position the flexible guide 32 against the interior of the stomach 10 using suction. As few as one port can be included, but generally the ports 38 comprise a plurality of longitudinally-spaced ports to provide suction along the length of the desired tissue fold 24. FIG. 3 shows this embodiment of the gastric plication guide positioned within the stomach before suction has been used to temporarily adhere the flexible guide 32 against the stomach wall. As shown in FIG. 3, the securing device 34 is placed across the pyloric valve 16 to position the gastric plication guide, though it should be noted that in some embodiments a portion of the guide may extend past the pyloric valve 16.

The steps involved in using this embodiment of the gastric plication guide are shown in FIGS. 4 and 5. The gastric plication guide 30 is first inserted through the esophagus 12 and into the stomach 10 of a subject, and positioned by placing the securing device 34 of the guide adjacent to the pyloric valve 16 of the subject. A portion of the stomach 10 is then contacted with one or more ports 38 of the flexible guide 32, and suction is applied, as shown in FIG. 4(a), to adhere the guide to the stomach lining. The shaping balloon 36 is then partially inflated as shown in FIG. 4(b) to draw the initial suture points 18 together and infold a portion of the stomach 10 to form the initial tissue fold 24. A first line of sutures 22 is then sewn along the base of the initial tissue fold 24. After the surgeon has run the first line of sutures, the shaping balloon 36 is then fully inflated to provide a size corresponding to the desired final stomach size. As shown in FIG. 4(c), when the shaping balloon 36 is fully expanded, the tissue fold 24 is drawn further into the stomach by infolding an additional portion of the stomach 10 to form the final tissue fold 24. The final tissue fold 24 is then secured by sewing a second suture line 26 along the base of the final tissue fold. Once all of the sutures have been placed, the shaping balloon 36 is deflated, the securing device 34 is disengaged, and the gastric plication guide 30 is removed from the stomach of the subject. In some embodiments, the first and second lines of sutures are sewn laparoscopically.

Another embodiment of the gastric plication guide including a shaping balloon 36 is shown in FIG. 6. In this embodiment, the shaping balloon 36 further comprises a cylindrical central guide balloon 40 positioned on the outside of the shaping balloon 36, and two cylindrical peripheral guide balloons 42 and 44 positioned within the shaping balloon 36 on each side of and in parallel with the central guide balloon 40. The additional balloons are used to grab the tissue that is folded into the stomach to form the tissue fold 24. Rather than showing the entire gastric plication guide device, FIG. 6 focuses on the shaping balloon itself, and the roles played by the additional balloons in forming the tissue fold 24. However, it should be understood that the shaping balloon 36 is appropriately positioned on a gastric plication guide 30 so that it will inflate within the stomach when the gastric plication guide has been positioned using the securing device 34. FIG. 7 provides a flowchart calling out the specific steps involved in carrying out a gastric plication procedure using this embodiment of the invention.

The first step in using the multi-balloon embodiment of the gastric plication guide involves inserting the gastric plication guide 30 through the esophagus 12 and into the stomach 10 of the subject, and positioning the device by placing the securing device 34 adjacent to the pyloric valve 16 of the subject. The shaping balloon 36 is then inflated to a size corresponding to the desired final stomach size. As shown in FIG. 6(a), the central guide balloon 40 is then fully inflated, and the peripheral guide balloons 42 and 44 are partially inflated. Additional lines can be provided within the lumen 40 of the gastric plication guide 30 that are connected to an air source in order to inflate and deflate the central and peripheral guide balloons. Force is then applied between the two peripheral guide balloons 42 and 44 to cause a portion of the stomach to infold and form an initial tissue fold 24. For example, force can be manually applied by the surgeon. The tissue fold 24 is secured in place by fully inflating the peripheral guide balloons 42 and 44, as shown in FIG. 6(b). A first suture line 22 is then sewn along the base of the initial tissue fold 24. The central guide balloon 40 is then deflated, and the peripheral guide balloons 42 and 44 are partially deflated to encourage infolding of an additional portion of the stomach to form the final tissue fold 24, as shown in FIG. 6(c). The second suture line 26 is then sewn along the base of the final tissue fold 24. The shaping balloon 36 is then deflated and the gastric plication guide is withdrawn from the stomach of the subject. In some embodiments, the first and second suture lines are sewn laparoscopically.

In another aspect, the means for guiding stomach tissue of the gastric plication guide is a groove 50 sized to accommodate the tissue fold 24. In one embodiment, the gastric plication guide is a bougie 48 with a groove 50 running longitudinally approximately along the length of the bougie 48 that will sit within the stomach 10 during the gastric plication surgery. A bougie is a shaping device that generally does not include an internal lumen, although an internal lumen can be included connect to an air supply to inflate an anchoring balloon, if a balloon is used as the securing device 34. Prior art bougies used in bariatric surgeries generally are defined based on their diameter alone. When this embodiment of the gastric plication guide has been positioned within the stomach, the physician will push a portion of the stomach wall into the groove 50 from the outside of the stomach to form a tissue fold, with the groove governing the size of the fold to prevent it from being too small or too large.

In further embodiments of the gastric plication guide that includes a groove, the flexible guide further includes a lumen 54 and one or more ports 52 positioned within the groove 50 and in communication with the lumen 54. A single port can be used, but preferably the ports include a plurality of longitudinally-spaced ports that are positioned along the base of the groove 50 so that suction is provided along the length of the tissue fold 24. When used in surgery, the surgeon defines the suture line by suctioning stomach tissue into the groove of the gastric plication guide 30, thereby folding the stomach tissue into the stomach to form a tissue fold 24.

Methods of using the gastric plication guides shown in FIG. 8 are illustrated in schematic form in FIG. 9. The first step is to insert the gastric plication guide (30 or 48) through the esophagus 12 and into the stomach 10 of a subject. The securing device 34 is then placed adjacent to the pyloric valve 16 in order to correctly position the gastric plication guide. A portion of the stomach is then contacted with the groove 50 of the guide. In embodiments that include suction ports 52, suction may then be applied to the stomach 10 through the ports in order to encourage formation of a tissue fold within the groove 50. A portion of the stomach is then folded into the groove 50 to form an initial tissue fold 24. A first suture line 22 is then sewn along the base of the initial tissue fold 24. An additional portion of the stomach is then folded into the groove 50 of the gastric plication guide to form a final tissue fold 24, with the groove serving to limit the amount of stomach tissue included. Vacuum can also be applied through suction ports 52, if present, during this step to assist with lengthening of the tissue fold 24. A second suture line 26 is then sewn along the base of the final tissue fold 24. Finally, the gastric plication guide is withdrawn from the stomach of the subject.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A gastric plication guide device, comprising:
an elongated flexible guide,
a securing device positioned on the distal end of the guide, and
means for guiding stomach tissue during a gastric plication procedure, positioned longitudinally along the distal end of the guide and proximal to the securing device.

2. The gastric plication guide of claim 1, wherein the means for guiding stomach tissue comprise a shaping balloon.

3. The gastric plication guide of claim 2, wherein the flexible guide includes a lumen, wherein the shaping balloon is positioned along one side of the guide, and the lumen includes one or more ports in communication with the lumen positioned along the guide opposite from the shaping balloon.

4. The gastric plication guide of claim 3, wherein the ports comprise a plurality of longitudinally-spaced ports.

5. The gastric plication guide of claim 2, wherein the shaping balloon further comprises a cylindrical central guide balloon positioned on the outside of the shaping balloon, and two cylindrical peripheral guide balloons positioned within the shaping balloon on each side of and in parallel with the central guide balloon.

6. The gastric plication guide of claim 1, wherein the means for guiding stomach tissue comprise a groove sized to accommodate folded stomach tissue.

7. The gastric plication guide of claim 6, wherein the flexible guide includes a lumen and one or more ports positioned within the groove and in communication with the lumen.

8. The gastric plication guide of claim 7, wherein the ports comprise a plurality of longitudinally-spaced ports positioned along the base of the groove.

9. A method of guided gastric plication, comprising the steps of:
inserting a gastric plication guide device according to claim 2 through the esophagus and into the stomach of the subject,
placing the securing device of the guide adjacent to the pyloric valve of the subject,
infolding a portion of the stomach to form an initial tissue fold,
sewing a first suture line along the base of the initial tissue fold,
infolding an additional portion of the stomach to form a final tissue fold,
sewing a second suture line along the base of the final tissue fold, and
withdrawing the gastric plication guide device from the stomach of the subject, wherein the method further comprises the step of inflating the shaping balloon within the stomach to a size corresponding to the desired final stomach size subsequent to the guided gastric plication, and deflating the shaping balloon before withdrawing the gastric plication guide device.

10. The method of claim 9, wherein the shaping balloon further comprises a cylindrical central guide balloon positioned on the outside of the shaping balloon, and two cylindrical peripheral guide balloons positioned within the shaping balloon on each side of and in parallel with the central guide balloon, wherein the shaping balloon is inflated to a size corresponding to the desired final stomach size after placing the securing device of the guide adjacent to the pyloric valve of the subject, and further comprising the steps of:
fully inflating the central guide balloon and partially inflating the peripheral guide balloons before infolding a portion of the stomach to form an initial tissue fold,
fully inflating the peripheral guide balloons to secure the initial tissue fold before sewing a first suture line along the base of the initial tissue fold,
fully deflating the central guide balloon and partially deflating the peripheral guide balloons before infolding an additional portion of the stomach to form a final tissue fold, and fully inflating the peripheral guide balloons to secure the final tissue fold before sewing a second suture line along the base of the final tissue fold.

11. The method of claim 10, wherein the first and second suture lines are sewn laparoscopically.

12. The method of claim 9, wherein the flexible guide includes a lumen, wherein the shaping balloon is positioned along one side of the guide, and the lumen includes one or more ports in communication with the lumen positioned along the guide opposite from the shaping balloon, and the method further comprises the steps of:
contacting a portion of the stomach with the one or more ports of the flexible guide and applying suction through the ports before infolding a portion of the stomach to form the initial tissue fold,
partially inflating the shaping balloon to assist formation of the initial tissue fold,
applying suction through the ports before infolding an additional portion of the stomach to form the final tissue fold, and fully inflating the shaping balloon to correspond to the desired final stomach size to assist formation of the final tissue fold.

13. The method of claim 12, wherein the first and second suture lines are sewn laparoscopically.

14. A method of guided gastric plication, comprising the steps of:
inserting a gastric plication guide device according to claim 6 through the esophagus and into the stomach of a subject,
placing the securing device of the guide adjacent to the pyloric valve of the subject and contacting a portion of the stomach with the groove of the flexible guide,
infolding a portion of the stomach into the groove to form an initial tissue fold, sewing a first suture line along the base of the initial tissue fold,
infolding an additional portion of the stomach into the groove of the guide device to form a final tissue fold,
sewing a second suture line along the base of the final tissue fold, and
withdrawing the gastric plication guide device from the stomach of the subject.

15. The method of claim 14, wherein the flexible guide includes a lumen and one or more ports positioned within the groove and in communication with the lumen, and the method further comprises applying suction to the stomach through the ports before infolding a portion of the stomach into the groove to form the initial tissue fold and/or the final tissue fold.

16. The method of claim 14, wherein the first and second suture lines are sewn laparoscopically.
